(19) United States
(12) Patent Application Publication
(10) Pub. No.: US 2010/0305202 A1
(43) Pub. Date: Dec. 2, 2010

(54) LYOPHILIZED PHARMACEUTICAL COMPOSITION WITH IMPROVED STABILITY CONTAINING TAXANE DERIVATIVES, AND METHOD OF MANUFACTURING THE SAME

(76) Inventors: Yong Youn Hwang, Gyeonggi-do (KR); Woo Jae Jang, Gyeonggi-do (KR); Joon-Gyo Oh, Gyeonggi-do (KR); Nam Ho Kim, Gyeonggi-do (KR); Jae-Sun Kim, Gyeonggi-do (KR); Key An Um, Gyeonggi-do (KR)

Correspondence Address: FROMMER LAWRENCE & HAUG, 745 FIFTH AVENUE- 10TH FL., NEW YORK, NY, 10151, US

(21) Appl. No.: 12/744,164

(22) PCT Filed: Nov. 21, 2008
(86) PCT No.: PCT/KR08/06875

§ 371 Date: Jun. 28, 2010

(30) Foreign Application Priority Data
Nov. 22, 2007 (KR) 10-2007-0119482

(51) Int. Cl. A61K 31/337; A61P 35/00

(52) U.S. Cl. 514/449

(57) ABSTRACT

The present invention relates to a lyophilized pharmaceutical composition for injection having superior storage stability comprising a taxoid, and a method thereof. More specifically, the present invention relates to a lyophilized pharmaceutical composition for injection having improved solubility and stability of dilution compared to the conventional preparations by dissolving a water-insoluble taxoid in distilled water added with a hydrophilic polymer such as hydroxypropylmethyl cellulose (HPMC), polyethylene glycol (PEG) or polyvinylpyrrolidone (PVP) cyclodextrin (CD), and lyophilizing the mixture and a method thereof.

### TECHNICAL FIELD

The present invention relates to a lyophilized pharmaceutical composition for injection containing a taxoid having superior stability. The invention further relates to a method of preparation thereof.

### BACKGROUND ART

Docetaxel is commercially available as a drug named Taxotere® having therapeutic effects against breast, ovarian, non-small cell lung, head and neck cancers. Docetaxel is a semi-synthetic taxoid with highly lipophilic and water-insoluble properties. Taxotere® is currently distributed in a blister carton package consists of one single-dose solution containing docetaxel dissolved in polysorbate 80 vial and one single-dose solvent for Taxotere containing 13% (w/w) ethanol vial. In use, the two are mixed together to obtain a premix solution having 10 mg/mL of docetaxel solubility, diluted in a saline solution or 5% dextrose solution to obtain an infusion solution having 0.3-0.74 mg/mL of docetaxel solubility, and then infused into blood veins for an hour. Thus prepared premix solution has poor storage stability allowing maximum storage of 8 hours when stored at room temperature or in a cold room. Further, the infusion solution should be used within 4 hours at 2-25° C. after its preparation, and any particles or precipitates should be observed by naked eye. In addition, hypersensitive reactions may occur because of using polysorbate 80 and side effects may occur due to the presence of ethanol.

WO 98/30205 discloses a method of using PEGylated Vitamin E as a surfactant, and USA 2004/0127551 discloses a method of using Vitamin E TPGS (d-alpha-tocopheryl polyethylene glycol 1000 succinate), however, both failed to provide a composition stably containing docetaxel with a high concentration range.

Korean Pat. No. 310839 discloses a method of preparing an injectable solution by mixing a paclitaxel and polyvinylpyrrolidone to obtain a matrix and then mixing the matrix with solvents such as anhydrous ethanol, polyoxyethylene glycerol ricinolate, polysorbate 80, polyethylene glycol, etc. However, this method also has a drawback that it contains substances causing alcohol addiction or hypersensitive reactions as is the case with ethanol and polysorbate 80.

WO 99/24073 filed in 1997 discloses a method of improving aqueous solubility of docetaxel by using cyclodextrin instead of using the above-mentioned surfactant. More specifically, docetaxel was dissolved in a small amount of ethanol, and thus obtained solution was added into acetyl gamma cyclodextrin (Ac-gamma-CD) or 5% dextrose solution of hydroxypropylmethyl β-cyclodextrin (HP-beta-CD). After removing most of the ethanol by evaporation or other appropriate methods, the resultant was lyophilized. It teaches that a preferable weight ratio of docetaxel to cyclodextrin is 1:25-1:400. The concentration of perfusate obtained by diluting the lyophilized composition above in 5% dextrose solution was 0.3-1.2 mg/mL and found to have more than 72 hours of physical stability.

However, the above invention also has problems that the ethanol used in the process of dissolving docetaxel may remain; the liquid phase composition obtained above may generate precipitates if the concentration of docetaxel is low; the physical stability of the lyophilized compound may be decreased if it is used after dissolution or dilution. Further, because it is difficult for the lyophilized composition to have 10 mg/mL of solubility, equivalent to that of Taxotere® premix solution, it is difficult to prepare a solution with concentration suitable for the clinical administration.

Another available taxoid is paclitaxel, widely known as Taxol, which has superior therapeutic effects against malignant tumors, in particular against breast, ovarian, non-small cell lung cancers. Unfortunately, the aqueous solubility of paclitaxel is so low that it is necessary to prepare mixed compositions for an injectable preparation by using a surfactant or ethanol as a base material. Ethanol is the best known solvent for solubilizing the paclitaxel.

According to the article by Rowinsky, Lorraine Cazenave & Donehower in the ^{┌}Journal of the National Cancer Institute_{┘} (p. 1247-1529, article 15, volume 82, Aug. 1, 1990), a mixed solution comprising ethanol and Cremophor EL added with Taxol (approx. 6 mg/mL) is prepared in advance and is mixed with perfusate containing dextrose or a saline solution at the time of injection. In order to obtain a stable composition from the physical and chemical point of view, it is necessary to limit the concentration of the active ingredient in the perfusate at 0.3-0.6 mg/mL.

For the effective anti-cancer treatment, it is recommended that a sufficient amount of an active ingredient be contained in the injection, preferably 0.3-1 mg/mL. If the injection contains more than the above amount (more than 1 mg/mL) uncontrollable anaphylactic shock may occur due to the Cremophor. Further, the above article discloses that a considerable amount of ethanol is administered for the injection of active ingredients at the concentration mentioned above, which may result in alcohol addiction.

Therefore, there has been a demand for development of a novel lyophilized composition for injection containing a taxoid, having improved storage stability and solubility after dilution compared to those of known compositions, and free of a harmful solubilizing agent to injection such as polysorbate or ethanol.

### DETAILED DESCRIPTION OF INVENTION

### Technical Problem

The present inventors made various efforts to solubilize and stabilize a water-insoluble taxoid in an aqueous solution, and finally succeeded in preparing a lyophilized composition for injection containing a taxoid having improved stability and solubility compared to the conventional preparations by dissolving hydroxypropyl β-cyclodextrin (HPBCD), and a hydrophilic polymer such as hydroxypropylmethyl cellulose (HPMC), polyethylene glycol (PEG) or polyvinylpyrrolidone (PVP) in distilled water. Accordingly, the object of the invention is to provide a lyophilized pharmaceutical composition for injection containing a taxoid having superior storage stability and a preparation method thereof.

### Technical Solution

The present invention relates to a lyophilized pharmaceutical composition for injection having superior storage stability comprising a water-insoluble taxoid, cyclodextrin, and a hydrophilic polymer such as hydroxypropylmethyl cellulose (HPMC), polyethylene glycol (PEG) or polyvinylpyrrolidone (PVP).

The invention further relates to a method of preparing the lyophilized composition for injection containing the taxoid having superior storage stability, which comprises:

1) dissolving a taxoid, cyclodextrin, and a hydrophilic polymer such as hydroxypropylmethyl cellulose (HPMC), polyethylene glycol (PEG) or polyvinylpyrrolidone (PVP) in distilled water;

2) lyophilizing the mixture obtained in step 1).

### BEST MODE

The present invention relates to solubilization of a water-insoluble taxoid by using cyclodextrin, and dissolving the taxoid in distilled water added with a hydrophilic polymer such as hydroxypropylmethyl cellulose (HPMC), polyethylene glycol (PEG) or polyvinylpyrrolidone (PVP). The present invention is characterized in preparing a lyophilized pharmaceutical composition for injection containing a high concentration taxoid without using an additive such as ethanol or polysorbate which often causes side-effects.

The present invention also relates to a method of preparing a lyophilized composition for injection containing a taxoid. In the method:

1) the first step is dissolving the taxoid, cyclodextrin, and hydrophilic polymers in distilled water. The water-insoluble taxoid is preferably a derivative as represented by the following Formula 1;

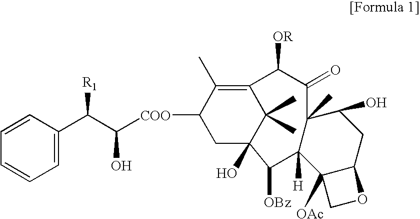

wherein R is hydrogen or an acetyl group, R₁ is a tertiary-butoxycarbonylamino radical or a benzoylamino radical.

The taxoid represented by the Formula 1 is preferably docetaxel wherein R is hydrogen, and R₁ is a tertiary-butoxycarbonylamino radical; or paclitaxel wherein R is an acetyl group, and R₁ is a benzoylamino radical.

Further, the taxoid is in a free form or in a form of pharmaceutically acceptable salts, anhydrous or hydrate thereof in the present invention. The preferable quantity of the taxoid is 0.2-50 wt. % in the total composition.

Cyclodextrins are classified upon their properties and pore sizes as α-cyclodextrin, β-cyclodextrin, and γ-cyclodextrin. The available cyclodextrin in the invention includes derivatives of cyclodextrin, preferably β-cyclodextrin or derivatives thereof, having 6.0-6.5 Å of diameter for each pore size, and more preferably hydroxypropyl β-cyclodextrin (HPBCD). Cyclodextrin is contained preferably 1-500 parts by weight relative to 1 part by weight of the taxoid, more preferably 5-200 parts by weight, and most preferably 5-100 parts by weight. If cyclodextrin is used too much, the resulting liquid composition becomes too viscous and it becomes difficult to filter it with 0.22 micrometer filter paper and to reconstitute final lyophilized composition by using a diluent before injection. On the contrary, if cyclodextrin is used too little, appropriate solubility and stability of the taxoid may not be obtained.

The degree of molecular substitution of hydroxypropyl β-cyclodextrin (HPBCD) is preferably 0.2-1.0, and more preferably 0.4-1.0. If the degree of molecular substitution is too low, the solubility of HPBCD becomes low. In contrast, if it is too high, HPBCD becomes too viscous too handle.

The hydrophilic polymer used in the present invention increases the stability of the taxoid in solution, and increases solubility of the taxoid by reacting with cyclodextrin.

Examples of the common hydrophilic polymers include polyethylene glycol (PEG), polyvinylpyrrolidinone (PVP), carboxymethylcellulose (CMC), hydroxypropylcellulose (HPC), hydroxymethylcellulose (HMC), hydroxyethylcellulose (HEC), hydroxypropylmethyl cellulose (HPMC), hydroxypropylethyl cellulose (HPEC), etc., and preferable hydrophilic polymers in the present invention is hydroxypropylmethyl cellulose (HPMC), polyethylene glycol (PEG) or polyvinyl pyrrolidone (PVP).

The viscosity of hydroxypropylmethyl cellulose (HPMC) is preferably 5-100,000 cps, and more preferably 5-4,000 cps. If the viscosity of hydroxypropylmethyl cellulose (HPMC) is too low, the solubility or stability of taxoid become remarkably poor. If the viscosity is too high, it is difficult to be treated and developed as an injection.

For the polyethylene glycol, there are various products with average molecular weight of 300-150,000. The preferable products of the polyethylene glycol are 300, 400, and 600 in their average molecular weight which are acceptable as an injection.

Further, the K-value of polyvinylpyrrolidone is preferably in the range of 10-20. If it is below 10, the solubility or stability of taxoid becomes remarkably poor. Meanwhile, if it is over 20, viscosity increases and it is difficult to be used as an injection.

The content of the hydrophilic polymers is preferably 0.01-100 parts by weight, and more preferably 0.1-10.0 parts by weight relative to 1 part by weight of the taxoid. If it is below 0.01 parts by weight, the solubility and stability of taxoid becomes remarkably low, if it is over 100, viscosity increases and filtering, cleaning, and using it as an injection becomes difficult.

Further, the solution used in the present invention is preferably any solution which acts as a perfusate, and more preferably distilled water for injection.

The method of dissolving the ingredients in distilled water in the invention is to dissolve the taxoid, cyclodextrin, and hydrophilic polymers in the amount to have the sufficient volume and concentration thereof for lyophilization in distilled water. The other method is a two-step dissolving process. The first step is dissolving them in a small amount of distilled water, and the second step is dissolving them again in distilled water added in the amount to an appropriate quantity for lyophilization.

Cyclodextrin plays an important role in the solubilization of the water-insoluble taxoid and the solubility is proportional to the aqueous concentration of cyclodextrin. In this regard, when the amount of cyclodextrin used is relatively small, a 2-step of dissolving method is preferable, wherein the amount of distilled water used in the first step is preferably to reach the appropriate concentration of cyclodextrin for solubilizing the taxoid.

According to the Experimental Examples in the present invention, even if the distilled water for injection added in the second step for lyophilization lowers the concentration of hydroxypropyl β-cyclodextrin (HPBCD), the water-insoluble taxoid does not precipitate and maintain the state of transparent dissolution.

The appropriate concentration of the taxoid for lyophilization is about 1.5-30 mg/mL. If it is lower than 1.5 mg/mL, the productivity of the lyophilizer decreases and the unit price of manufacturing increases. If it is greater than 30 mg/mL, the solubility of the taxoid does not improve, but the viscosity increases, thus making it difficult to conduct a commercial sterilization.

2) the second step is heating, stirring and lyophilizing the mixture obtained in the above step. The stirring is performed at a temperature of 5-50° C., preferably 15-30° C. The resultant is frozen for the lyophilization at between −80 and −30° C. under the reduced pressure, and then a white or light yellow lyophilized composition is obtained.

Additionally, in order to use the lyophilized composition for injection, the lyophilized composition should be diluted to obtain a premix solution. The diluent may be any injectable solutions, and preferably water for injection, dextrose solution or a saline solution. The degree of dilution may be various depending upon the preparation method of liquid composition. The concentration of the taxoid is preferably 1-20 mg/mL. The premix solution prepared is diluted in a saline solution or 5% dextrose solution and injected into a vein as in a conventional way.

The lyophilized pharmaceutical composition obtained according to the present invention achieves excellent stability which is not affected by temperature and humidity. Thus it may be stored for a long time, easily prepared for a formulation for an injection, and not decomposed by the temperature and humidity conditions during the manufacturing process.

Further, it can be safely administered to humans without ethanol, Cremophor or any additives, which may cause hypersensitive side effects.

The present invention may be further described with the Examples herein after but the invention is not limited to these.

### Experimental Example 1

Polyvinylpyrrolidone (PVP, K-12), and hydroxypropyl β-cyclodextrin (HPBCD) (MS=0.6) were weighed as shown in Table 1, excessive anhydrous docetaxel was added, and then mixed with distilled water to make the mixtures to 10 mL, stirred for 4 days, filtered through 0.22 micrometer syringe filter. The solubility of docetaxel was measured by performing liquid chromatography with UV detector (230 nm).

The result of the Experimental Example shows that the commonly known solubility of docetaxel in distilled water is 0.000025 mg/mL, whereas the solubility of docetaxel added with hydroxypropyl β-cyclodextrin (HPBCD) in distilled water is 31.1 mg/mL, which means 1.2 million times better solubility of the latter as well as superior solubility surpassing 10 mg/mL of concentration of docetaxel in premix solution of Taxotere®. The solubility of docetaxel in distilled water proportionally increases between 10% and 30% of weight ratio (w/v %) of hydroxypropyl β-cyclodextrin (HPBCD).

Further, the ratio of HPBCD relative to docetaxel was at least 1:11 and the concentration of HPBCD will be lowered if the concentration unit (w/v) is converted into (mg/mL) unit. Therefore, docetaxel may be solubilized even when the ratio of docetaxel:HPBCD becomes smaller than 1:11. Therefore, according to the present invention, it is preferable to use more than 1 part by weight of hydroxypropyl β-cyclodextrin (HPBCD), and more preferably 5 parts by weight relative to 1 part by weight of the taxoid.

TABLE 1

| | | | | Solubility | |
|---|---|---|---|---|---|
| | PVP | HPBCD | HPBCD | of Docetaxel | Docetaxel: |
| No. | (g/10 mL) | (g/10 mL) | (w/v %) | (mg/mL) | HPBCD |
| 1-1 | 0.075 | 1.0 | 10% | 0.9 | 1:114 |
| 1-2 | 0.075 | 2.0 | 20% | 15.4 | 1:13 |
| 1-3 | 0.075 | 3.0 | 30% | 27.7 | 1:11 |
| 1-4 | 0.075 | 4.0 | 40% | 31.1 | 1:13 |
| 1-5 | 0.075 | 5.0 | 50% | 30.9 | 1:16 |

### Experimental Example 2

Anhydrous docetaxel, polyvinylpyrrolidone (PVP K-12), and hydroxypropyl β-cyclodextrin (HPBCD) (MS=0.6) were weighed to have the concentration as shown in Table 2, added with distilled water for injection, stirred and dissolved, and after 8 hours from the completion, the solution was filtered through 0.22 micrometer syringe filter, analyzed with HPLC, and then the ratio of peak area (%) of related substances of docetaxel was calculated.

The result of the above Experimental Examples shows that related substances of docetaxel are remarkably reduced in the solution containing a hydrophilic polymer of polyvinylpyrrolidone, which is effective in stabilizing docetaxel in an injectable solution.

TABLE 2

| | Anhydrous | Polyvinyl | Hydroxypropyl | Ratio of Area of |
|---|---|---|---|---|
| | docetaxel | Pyrrolidone | β-Cyclodextrin | Related Substances |
| No. | (mg/mL) | (mg/mL) | (mg/mL) | of Docetaxel (%) |
| 2-1 | 10 | 0 | 150 | 9.5 |
| 2-2 | 10 | 10 | 150 | 1.6 |
| 2-3 | 10 | 30 | 150 | 0.0 |
| 2-4 | 10 | 0 | 200 | 6.4 |
| 2-5 | 10 | 10 | 200 | 1.2 |
| 2-6 | 10 | 30 | 200 | 0.5 |

According to Experimental Example 1 and 2, the composition does not contain ethanol or a surfactant but comprises a taxoid, hydroxypropyl β-cyclodextrin (HPBCD), and a hydrophilic polymer. The taxoid is preferably contained in the range of 0.2-50 wt. %, and more preferably 0.5-20 wt. %.

### Example 1

Anhydrous docetaxel (100 mg), polyvinylpyrrolidone K-12 (100 mg), and hydroxypropyl β-cyclodextrin (HPBCD) (MS=0.6, 1500 mg) were weighed and dissolved in distilled water for injection to a final volume of 5 mL of a transparent solution. The concentration of docetaxel in this step was 20 mg/mL. Upon completion of the reaction, distilled water for injection was added to a final volume of 20 mL, and then thus obtained solution was filtered through 0.22 micrometer filter, and the resultant was cooled at around −45° C., and then lyophilized to obtain a lyophilized composition.

### Example 2

Anhydrous docetaxel (100 mg), polyvinylpyrrolidone K-12 (100 mg), and hydroxypropyl β-cyclodextrin (HPBCD) (MS=0.6, 2000 mg) were weighed and dissolved in distilled water for injection to a final volume of 7 mL of a transparent solution. The concentration of docetaxel in this step was 14.3 mg/mL. Upon completion of the reaction, distilled water for injection was added to a final volume of 20 mL, filtered through 0.22 micrometer filter, and the resultant was cooled at around −45° C., and then lyophilized to obtain a lyophilized composition.

### Example 3

Anhydrous docetaxel (100 mg), polyvinylpyrrolidone K-12 (300 mg), and hydroxypropyl β-cyclodextrin (HPBCD) (MS=0.6, 1500 mg) were weighed and dissolved in distilled water for injection to a final volume of 7 mL of a transparent solution. The concentration of docetaxel in this step was 20 mg/mL. Upon completion of the reaction, distilled water for injection was added to a final volume of 20 mL, filtered through 0.22 micrometer filter, and the resultant was cooled at around −45° C., and then lyophilized to obtain a lyophilized composition.

### Example 4

Anhydrous docetaxel (100 mg), polyvinylpyrrolidone K-12 (300 mg), and hydroxypropyl β-cyclodextrin (HPBCD) (MS=0.6, 2000 mg) were weighed and dissolved in distilled water for injection to a final volume of 7 mL of a transparent solution. The concentration of docetaxel in this step was 14.3 mg/mL. Upon completion of the reaction, distilled water for injection was added to a final volume of 20 mL, filtered through 0.22 micrometer filter, and the resultant was cooled at around −45° C., and then lyophilized to obtain a lyophilized composition.

### Example 5

Anhydrous docetaxel (100 mg), polyvinylpyrrolidone K-12 (300 mg), and hydroxypropyl β-cyclodextrin (HPBCD) (MS=0.6, 2500 mg) were weighed and dissolved in distilled water for injection to a final volume of 7 mL of a transparent solution. The concentration of docetaxel in this step was 14.3 mg/mL. Upon completion of the reaction, distilled water for injection was added to a final volume of 20 mL, and then thus obtained solution was filtered through 0.22 micrometer filter, and the resultant was cooled at around −45° C., and then lyophilized to obtain a lyophilized composition.

### Example 6

Anhydrous docetaxel (100 mg), polyvinylpyrrolidone K-12 (300 mg), and hydroxypropyl β-cyclodextrin (HPBCD) (MS=0.6, 5000 mg) were weighed and dissolved in distilled water for injection to a final volume of 10 mL of a transparent solution. The concentration of docetaxel in this step was 10 mg/mL. Upon completion of the reaction, distilled water for injection was added to a final volume of 20 mL, and then thus obtained solution was filtered through 0.22 micrometer filter, and the resultant was cooled at around −45° C., and then lyophilized to obtain a lyophilized composition.

### Example 7

Anhydrous docetaxel (100 mg), polyvinylpyrrolidone K-12 (300 mg), and hydroxypropyl β-cyclodextrin (HPBCD) (MS=0.6, 6000 mg) were weighed and dissolved in distilled water for injection to a final volume of 20 mL of a transparent solution. The concentration of docetaxel in this step was 5 mg/mL. Then, thus obtained solution was filtered through 0.22 micrometer filter, and the resultant was cooled at around −45° C., and then lyophilized to obtain a lyophilized composition.

### Example 8

Anhydrous docetaxel (100 mg), hydroxypropylmethyl cellulose (50 cps, 100 mg), and hydroxypropyl β-cyclodextrin (HPBCD) (MS=1.0, 3000 mg) were weighed and dissolved in distilled water for injection to a final 10 mL of a transparent solution. The concentration of docetaxel in this step was 10 mg/mL. Upon completion of the reaction, distilled water for injection was added to a final volume of 20 mL of the solution, and then thus obtained solution was filtered through 0.22 micrometer filter, and the resultant was cooled at around −45° C., and then lyophilized to obtain a lyophilized composition.

### Example 9

Anhydrous docetaxel (100 mg), polyethylene glycol (M.W. 400, 100 mg), and hydroxypropyl β-cyclodextrin (HPBCD) (MS=0.6, 3000 mg) were weighed and dissolved in distilled water for injection to a final volume of 10 mL of a transparent solution. The concentration of docetaxel in this step was 10 mg/mL. Upon completion of the reaction, distilled water for injection was added to a final volume of 20 mL, and then thus obtained solution was filtered, and the resultant was cooled at around −45° C., and then lyophilized to obtain a lyophilized composition.

### Example 10

Paclitaxel (100 mg), polyvinyl pyrrolidone K-12 (300 mg), and hydroxypropyl β-cyclodextrin (HPBCD) (MS=0.6, 2000 mg) were weighed and dissolved in distilled water for injection to a final volume of 7 mL of a transparent solution. The concentration of paclitaxel in this step was 14.3 mg/mL. Upon completion of the reaction, distilled water for injection was added to a final volume of 20 mL, and then thus obtained solution was filtered through 0.22 micrometer filter, and the resultant was cooled at around −45° C., and then lyophilized to obtain a lyophilized composition.

### Experimental Example 3

The composition obtained in the Example 4, 5, and the Taxotere® presently available in the market, were stored at cold temperature (4° C.) and at room temperature (25° C.) for a month, and then the ratio of peak area of related substances was analyzed with HPLC.

The result of the present test shows that the lyophilized composition according to the Examples 4 and 5 has superior storage stability compared to the commercially available product.

TABLE 3

| | Cold Storage Temperature | Room Temperature |
|---|---|---|
| Category | (4° C.) | (25° C.) |
| Example 4 | 0.18 | 0.22 |
| Example 5 | 0.23 | 0.26 |
| Taxotere ® | 0.65 | 0.81 |

### Experimental Example 4

The lyophilized composition obtained in Example 7 was diluted in 5% dextrose solution and a premix solution of Taxotere® was also prepared to have 0.7 mg/mL of concentration of docetaxel. Both of the solutions were stored at room temperature of 25° C. for 8 hours, and then docetaxel content, related substances and appearance of the solutions were confirmed.

The result of the test in the present invention shows that the lyophilized composition according to Example 7 has superior storage stability in dilution compared to the commercially available product.

TABLE 4

| | Contents Relative | Ratio of Peak Area for | |
|---|---|---|---|
| Category | to Initial Content | Total Related Substances | Appearance |
| Example 7 | 99.9% | 0.2 | Solution |
| | | | Transparent |
| | | | [symbol] |
| Taxotere ® | 77.3% | 0.8 | Solution |
| | | | Precipitated |
| | | | [symbol] |

### Examples 11-22

Docetaxel trihydrate (32 mg), anhydrous docetaxel (30 mg), hydroxypropylmethyl cellulose (HPMC), polyvinylpyrrolidone (PVP), or hydroxypropyl β-cyclodextrin (HPBCD) were weighted as shown in Table 5, dissolved in distilled water for injection, stirred and dissolved at room temperature, filtered through 0.22 micrometer filter, and then sterilized. The solubility of the resultant solution was measured, cooled at −80° C., and then lyophilized.

TABLE 5

| Category | | Ex. 11 | Ex. 12 | Ex. 13 | Ex. 14 | Ex. 15 | Ex. 16 |
|---|---|---|---|---|---|---|---|
| Docetaxel Trihydrate | | 96 | 96 | 96 | 96 | 96 | 96 |
| HPBCD | M = 0.6 | 4500 | — | 9000 | — | 6300 | — |
| (mg) | M = 1.0 | — | 4500 | — | 9000 | — | 6300 |
| HPMC | 5 cps | 90 | 450 | — | — | 90 | — |
| (mg) | 100 cps | — | — | 90 | 360 | — | 450 |
| Distilled water for injection (mL) | | 18 | 18 | 9 | 9 | 24 | 24 |
| Solubility (mg/mL) | | 3.9 | 3.8 | 5.2 | 5.1 | 3.0 | 3.1 |
| Category | | Ex. 17 | Ex. 18 | Ex. 19 | Ex. 20 | Ex. 21 | Ex. 22 |
| Docetaxel Trihydrate | | 96 | 96 | 96 | 96 | 96 | 96 |
| HPBCD | M = 0.6 | 4500 | — | 9000 | — | 6300 | — |
| (mg) | M = 1.0 | — | 4500 | — | 9000 | — | 6300 |
| PVP(mg) | K-12 | 90 | 450 | — | — | 90 | — |
| | K-17 | — | — | 90 | 360 | — | 450 |

| | | | | | | |
|---|---|---|---|---|---|---|
| Distilled water for injection (mL) | 9 | 9 | 9 | 9 | 9 | 9 |
| Solubility (mg/mL) | 6.6 | 6.8 | 5.3 | 5.2 | 5.4 | 5.5 |

### Examples 23-34

Anhydrous docetaxel (30 mg), polyvinyl pyrrolidone (PVP), HPMC, polyethylene glycol (PEG), or hydroxypropyl β-cyclodextrin (HPBCD) were weighed as shown in Table 6, stirred and dissolved in distilled water for injection at room temperature, filtered through 0.22 micrometer filter, and then sterilized. The solubility of the resultant solution was measured, cooled at −80° C., and then lyophilized to obtain a lyophilized composition.

TABLE 6

| Category | | Ex. 23 | Ex. 24 | Ex. 25 | Ex. 26 | Ex. 27 | Ex. 28 |
|---|---|---|---|---|---|---|---|
| Docetaxel Trihydrate | | 90 | 90 | 135 | 135 | 90 | 90 |
| HPBCD | M = 0.6 | 4500 | — | 9000 | — | 6300 | — |
| (mg) | M = 1.0 | — | 4500 | — | 9000 | — | 6300 |
| PEG(mg) | MW400 | 90 | 450 | — | — | 270 | — |
| HPMC(mg) | 100 cps | — | — | 135 | 360 | — | 360 |

| Distilled water for injection (mL) | | 30 | 30 | 6 | 6 | 12 | 12 |
|---|---|---|---|---|---|---|---|
| Solubility (mg/mL) | | 2.6 | 2.7 | 8.9 | 9.2 | 4.9 | 5.0 |
| Category | | Ex. 29 | Ex. 30 | Ex. 31 | Ex. 32 | Ex. 33 | Ex. 34 |
| Docetaxel Trihydrate | | 90 | 90 | 135 | 135 | 90 | 90 |
| HPBCD | M = 0.6 | — | 4500 | 9000 | — | 6300 | — |
| (mg) | M = 1.0 | 4500 | — | — | 9000 | — | 6300 |
| PVP(mg) | K-12 | 90 | 450 | — | — | 270 | — |
| | K-17 | — | — | 45 | 120 | — | 360 |

| | | | | | | |
|---|---|---|---|---|---|---|
| Distilled water for injection (mL) | 6 | 6 | 6 | 6 | 12 | 12 |
| Solubility (mg/mL) | 8.6 | 8.7 | 9.0 | 9.2 | 5.0 | 4.9 |

### Comparative Example 1

A white lyophilized composition was obtained by using the same method as in the above Example 29, except for using a hydrophilic polymer.

### Comparative Example 2

A lyophilized composition containing docetaxel and HPBCD was prepared by using the same method as in Example I. 10 of WO 99/24073. Docetaxel (60 mg) was dissolved in ethanol (3 mL), added with HPBCD (3000 mg), and added with distilled water for injection (60 mL) to obtain a transparent solution having 1 mg/mL of concentration. The solution was quick-frozen using dry ice, lyophilized to obtain a lyophilized composition in a powdered form containing 2% w/w of docetaxel.

### Comparative Example 3

According to the prescription of the Taxotere® presently available in the market, a lyophilized composition was prepared using the same method as in the Korean Pat. No. 0136722. Docetaxel trihydrate (96 mg) was dissolved in anhydrous ethanol (1020 mL), added with polysorbate 80 (2490 mg), and then ethanol was evaporated under reduced pressure (30° C.) for 2 hours in a rotary evaporator.

### Experimental Example 1

### Stability Test (Liquid Form)

The lyophilized composition of Examples 11-34, and Comparative Example 1 and 3 were added with distilled water for injection to obtain a composition in liquid. Then, the stability at room temperature according to time passage was measured by HPLC based on the concentration relative to the initial concentration.

TABLE 7

| | Initial | Concentration | Concentration | Change in |
|---|---|---|---|---|
| | Concentration | (mg/mL) | (mg/mL) | Solution |
| Category | (mg/mL) | After 48 hrs. | After 96 hrs. | After 96 hrs. |
| Ex. 11 | 3.8 | 3.8 | 3.8 | clear solution |
| Ex. 13 | 5.1 | 5.1 | 5.0 | clear solution |
| Ex. 15 | 3.1 | 3.0 | 3.0 | clear solution |
| Ex. 17 | 6.7 | 6.6 | 6.5 | clear solution |
| Ex. 19 | 5.3 | 5.3 | 5.3 | clear solution |
| Ex. 21 | 5.3 | 5.3 | 5.2 | clear solution |
| Ex. 24 | 2.8 | 2.8 | 2.6 | clear solution |
| Ex. 26 | 9.1 | 9.1 | 9.1 | clear solution |
| Ex. 28 | 5.0 | 5.0 | 4.9 | clear solution |
| Ex. 30 | 8.8 | 8.7 | 8.6 | clear solution |
| Ex. 32 | 9.3 | 9.2 | 9.1 | clear solution |
| Ex. 34 | 4.8 | 4.8 | 4.7 | clear solution |
| Comp. | 8.6 | 5.2 | 1.9 | precipitation |
| Ex. 1 | | | | |

Test was conducted by dissolving the compositions obtained in Example 34 and Comparative Example 3 in 0.9% of a saline solution, and diluting to have 2.0 mg/mL of concentration.

TABLE 8

| | Initial | Concentration | Concentration | Change in |
|---|---|---|---|---|
| | Concentration | (mg/mL) | (mg/mL) | Solution |
| Category | (mg/mL) | After 10 hrs. | After 36 hrs. | After 36 hrs. |
| Comp. Ex. 3 | 2.0 | 1.8 | 1.2 | precipitation |
| Diluted | 1.9 | 1.9 | 1.8 | clear solution |
| Composition | | | | |
| of Ex. 34 | | | | |

As shown in Table 8, lyophilized composition obtained in Examples 11-34 according to the present invention had superior storage stability relative to Comparative Example 1. Further, as shown in the above Table 8, stability of dilution was superior to that of Comparative Example 3.

### Experimental Example 6

### Stability Test (Dry Form)

Lyophilized compositions obtained in Examples 23, 33 and Comparative Examples 2, 3 were stored at cold-storage condition (4° C.), long-term storage condition (25° C., 60% RH), and acceleration condition (40° C., 75% RH; 50° C., 60% RH), and thereby the stability of the composition in those conditions was detected. Stability test was performed based on the amount of related substances, and the following Table 9 shows that the lyophilized composition of the present invention has superior storage stability relative to those in Comparative Examples 2 and 3.

TABLE 9

| Category | | Initial Phase | 2 weeks | 4 weeks |
|---|---|---|---|---|
| Ex. 23 | 4° C. | 0.13 | 0.17 | 0.31 |
| | 25° C., 60% RH | | 0.33 | 0.41 |
| | 40° C., 75% RH | | 0.71 | 1.74 |
| | 50° C., 60% RH | | 1.21 | 3.30 |
| Ex. 33 | 4° C. | 0.09 | 0.13 | 0.16 |
| | 25° C., 60% RH | | 0.17 | 0.20 |
| | 40° C., 75% RH | | 0.35 | 0.30 |
| | 50° C., 60% RH | | 0.43 | 0.53 |
| Comp. | 4° C. | 0.32 | 0.48 | 0.49 |
| Ex. 2 | 25° C., 60% RH | | 0.79 | 0.82 |
| | 40° C., 75% RH | | 2.02 | 3.21 |
| | 50° C., 60% RH | | 3.48 | 4.12 |
| Comp. | 4° C. | 1.84 | 2.09 | 2.66 |
| Ex. 3 | 25° C., 60% RH | | 4.67 | 5.43 |
| | 40° C., 75% RH | | 8.82 | 9.31 |
| | 50° C., 60% RH | | 15.28 | 15.74 |

### INDUSTRIAL APPLICABILITY

The lyophilized pharmaceutical composition for injection containing a taxoid according to the present invention may be prepared into a formulation with superior solubility of the taxoid by using only safe ingredients for the body.

The lyophilized pharmaceutical composition according to the present invention has the following advantages: it can maintain docetaxel content during the long-term storage and after its dilution; it generates little amount of related substances so that it can tolerate the changes in the temperature and humidity during the manufacturing process; and its therapeutic effect can be maintained after its dilution for administration or during storage. Further, the lyophilized pharmaceutical composition for injection containing a taxoid according to the invention does not contain ethanol, polysorbate or Cremophor and are thus free from any side effects resulting from these ingredients.

1. A lyophilized pharmaceutical composition comprising a water-insoluble taxoid, cyclodextrin, and at least one hydrophilic polymer selected from the group consisting of hydroxypropylmethyl cellulose (HPMC), polyethylene glycol (PEG), and polyvinylpyrrolidone (PVP).

2. The composition as claimed in claim 1, wherein said taxoid is represented by the following Formula 1; 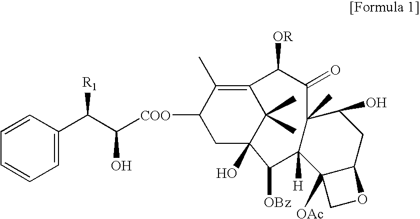
wherein R is a hydrogen atom or an acetyl group, R₁ is a tertiary-butoxy carbonylamino radical or a benzoylamino radical.

3. The composition as claimed in claim 2, wherein said taxoid is docetaxel represented by Formula 1, wherein R is a hydrogen atom, R₁ is a tertiary-butoxy carbonylamino radical.

4. The composition as claimed in claim 2, wherein said taxoid is paclitaxel represented by Formula 1, wherein R is an acetyl group and R₁ is a benzoylamino radical.

5. The composition as claimed in claim 2, wherein said taxoid is in a free form or in a form of pharmaceutically acceptable salts, anhydrous or hydrate thereof.

6. The composition as claimed in claim 1, wherein the cyclodextrin is 1-500 parts by weight relative to 1 part by weight of the taxoid.

7. The composition as claimed in claim 1, wherein the cyclodextrin is 5-200 parts by weight relative to 1 part by weight of the taxoid.

8. The composition as claimed in claim 1, wherein the hydrophilic polymers are 0.01-100 parts by weight relative to 1 part by weight of the taxoid.

9. The composition as claimed in claim 1, wherein the cyclodextrin is β-cyclodextrin or derivatives thereof.

10. The composition as claimed in claim 1, wherein the cyclodextrin is hydroxypropyl β-cyclodextrin.

11. The composition as claimed in claim 10, wherein the degree of molecular substitution (MS) of the hydroxypropyl β-cyclodextrin is 0.2-1.0.

12. The composition as claimed in claim 1, wherein the viscosity of the hydroxypropylmethyl cellulose (HPMC) is in the range of 5-100,000 cps.

13. The composition as claimed in claim 1, wherein the average molecular weight of the polyethylene glycol (PEG) is in the range of 300-600.

14. The composition as claimed in claim 1, wherein K-value of the polyvinylpyrrolidone is in the range of 10-20.

15. The lyophilized pharmaceutical composition for injection as claimed in claim 1, wherein the taxoid is contained in the range of 0.2-50 weight percent.

16. A method of preparing a lyophilized pharmaceutical composition containing a taxoid, which comprises:
1) dissolving a water-insoluble taxoid, cyclodextrin, and at least one hydrophilic polymer selected from the group consisting of hydroxypropylmethyl cellulose (HPMC), polyethylene glycol (PEG) or polyvinylpyrrolidone (PVP) in distilled water;
2) lyophilizing the mixture obtained in the step 1).

17. A method of preparing a pharmaceutical liquid composition for injection containing a taxoid, comprising:
1) dissolving a water-insoluble taxoid, cyclodextrin, and at least one hydrophilic polymer selected from the group consisting of hydroxypropylmethyl cellulose (HPMC), polyethylene glycol (PEG) or polyvinylpyrrolidone (PVP) in distilled water;
2) lyophilizing the mixture obtained in step 1); and
3) diluting the lyophilized composition obtained in step 2) in distilled water, dextrose solution or a saline solution.
